(19) United States
(12) Patent Application Publication
(10) Pub. No.: US 2011/0040377 A1
(43) Pub. Date: Feb. 17, 2011

(54) COMPOUND MICRO LENS IMPLANT

(75) Inventors: Robert Battis, Mercerville, NJ (US); John T. LiVecchi, Newport Richey, FL (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(73) Assignee: Laser Energies, Inc., Mercerville, NJ (US)

(21) Appl. No.: 12/866,362

(22) PCT Filed: Feb. 5, 2009
(86) PCT No.: PCT/US2009/033279

§ 371 Date: Nov. 3, 2010

Related U.S. Application Data
(60) Provisional application No. 61/026,293, filed on Feb. 5, 2008.

(51) Int. Cl. A61F 2/16

(52) U.S. Cl. 623/6.32

(57) ABSTRACT

A compound micro lens having a multiplicity of micro-lenses on a common substrate, with the micro-lenses having a multiplicity of focal lengths. Having a multiplicity of focal lengths allows the compound micro lens to be used in cataract surgery to replace human eye lens and still allow the patient to focus at different depths without the need for multiple pairs of glasses or other aids.

### CROSS REFERENCE TO APPLICATIONS

This application is related to, and claims priority from, U.S. Provisional Patent application No. 61/026,293 filed on Feb. 5, 2008, by R. Battis et al. titled “Compound Micro Lens Implant”, the contents of which are hereby incorporated by reference.

### TECHNICAL FIELD

The present invention relates to ophthalmic lenses, and more particularly, to multi-focal, compound micro-lens for replacement of human eye lenses and cornea.

### BACKGROUND ART

According to the World Health Organization, age-related cataracts are responsible for 48% of world blindness, which represents about 18 million people world wide. Cataracts cause the lens of the eye to become opaque.

FIG. 1 is a schematic representation of a human eye 10. The a human eye 10 has a lens 12, a cornea 14, an iris 16, and a retina 22. When functioning normally, light is focused by the cornea 14 and the lens 12 onto the retina 22 at the back of the eye. The information is then transmitted to the brain via the optic nerve 26. Although the cornea 14 supplies about two-thirds of the focal power of the a human eye 10, it has a fixed focus. The lens 12 is flexible and its shape can be changed by muscles 18 to provide variable focus for the eye.

The conventional surgery for cataract is to replace the lens 12 with a fixed gain synthetic lens. Although this restores the patients sight, it limits their ability to focus at different depths without the aid of glasses or contact lenses.

A synthetic lens that could be used to replace lenses made inoperable by cataracts and would allow the patient to focus at different distances is, therefore, a highly desirable device.

### SUMMARY OF THE INVENTION

The present invention is a compound micro lens that has a multiplicity of focal lengths and may therefore be used in cataract surgery to replace human eye lens and still allow the patient to focus at different depths without the need for multiple pairs of glasses.

Technical Problem

The technical problem addressed by the present invention includes the problem of how to provide a multiplicity of focal lengths with a solid lens.

Solution to Problem

The present invention solves the problem by providing a multiplicity of micro-lenses on a common substrate, with the micro-lenses having a multiplicity of focal lengths.

### ADVANTAGEOUS EFFECTS OF INVENTION

Advantages of the invention include, but are not limited to, the ability to use a single, solid lens to provide a multiplicity of focal lengths.

These and other features of the invention will be more fully understood by references to the following drawings.

### BRIEF DESCRIPTION OF DRAWINGS

FIG. 1 shows a schematic representation of a human eye.

FIG. 2A shows a plan view of a compound micro lens of the present invention.

FIG. 2B shows a sectional view of a compound micro lens of the present invention.

FIG. 3 shows a schematic view of the functioning of a three focal length compound micro lens of the present invention.

FIG. 4 that shows a compound micro lens have hexagonally shaped micro lenses.

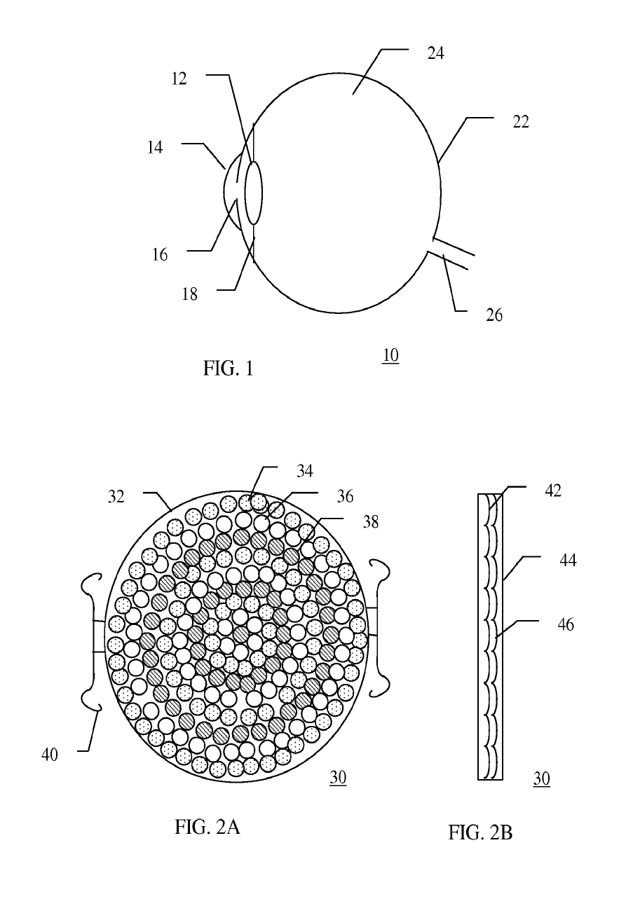

### DESCRIPTION OF EMBODIMENTS

Embodiments of the present invention will now be described in detail by reference to the accompanying drawings in which, as far as possible, like elements are designated by like numbers.

Although every reasonable attempt is made in the accompanying drawings to represent the various elements of the embodiments in relative scale, it is not always possible to do so with the limitations of two-dimensional paper. Accordingly, in order to properly represent the relationships of various features among each other in the depicted embodiments and to properly demonstrate the invention in a reasonably simplified fashion, it is necessary at times to deviate from absolute scale in the attached drawings. However, one of ordinary skill in the art would fully appreciate and acknowledge any such scale deviations as not limiting the enablement of the disclosed embodiments.

FIG. 2A shows a plan view of a compound micro lens 30 of the present invention. The compound micro lens 30 may have a substrate 32 on which there are a large number of micro lenses. The micro lenses may have a variety of focal lengths including, but not limited to, a short focal length micro lens 34, a medium focal length micro lens 36 and a long focal length micro lens 38. The various focal length lenses may for instance, but are not limited to, be arranged randomly, in grid patterns, or in circular patterns as shown in FIG. 2A. The compound micro lens 30 may also have side fixture elements 40 to hold them in place.

FIG. 2B show as sectional view of a compound micro lens of the present invention. The micro lens surface 42 may be covered by a cover material 44. The cover material 44 may have a cover material surface 46 that may match the micro lens surface 42 or may differ from the micro lens surface 42 in order to provide additional focal power. The cover material 44 may have a refractive index that matches the refractive index of the substrate 32 and micro lenses, or it may have a different refractive index to provide additional focal power.

FIG. 3 shows a schematic view of the functioning of a three focal length compound micro lens 30 of the present invention. The short focal length micro lenses 34 all focus a near by object 41 onto the retina 22. The medium focal length micro lenses 36 all focus medium distance objects 42 onto the retina 22. The long focal length micro lenses 38 all focus distant objects 44 on to the retina 22. In FIG. 2, the short focal length micro lens 34 and medium focal length micro lens 36 are shown to focus to a point centered on the optic axis 46 of the eye, while the long focal length micro lens 38 focuses to an off axis point 48. One of ordinary skill in the optical arts will realize that all lenses could focus to the point centered on the optic axis 46, or all could focus to the off axis point 48, or some combination thereof.

For instance the short focal length micro lens 34 may be selected to enable a patent to read, while said long focal length micro lens 38 may be selected to enable a patent to drive a car.

### EXAMPLES

### Example 1

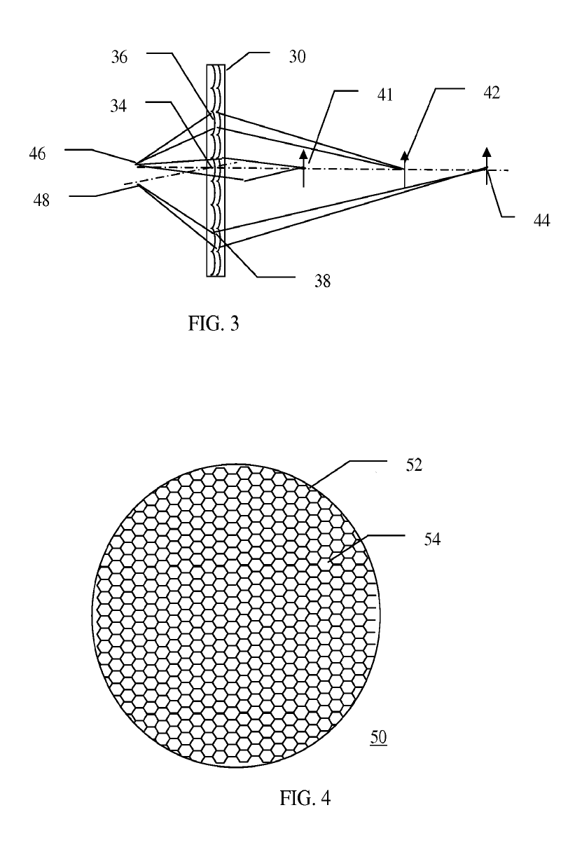

Example 1 is illustrated in, for instance, FIG. 4 that shows a compound micro lens have hexagonally shaped micro lenses. The individual micro lenses may, for instance, have a range of different shapes, including, but not limited to, circles, hexagons, polygons, partial circles, ellipses or other geometric shapes. The layout pattern, or centroids of the mico lenses may include, but are not limited to, nested rings, grid patterns, diamond patterns, random patterns or some combination thereof. The number of micro lenses in one compound lens may be as few as twenty-five or as many as several hundred, depending on the size of the micro lenses that may vary from a micron in diameter to several hundred microns in diameter.

The micro lens elements may be designed as negative lenses, positive lenses or some combination thereof.

Although the invention has been described in language specific to structural features and/or methodological acts, it is to be understood that the invention defined in the appended claims is not necessarily limited to the specific features or acts described. Rather, the specific features and acts are disclosed as exemplary forms of implementing the claimed invention. Modifications may readily be devised by those ordinarily skilled in the art without departing from the spirit or scope of the present invention.

### INDUSTRIAL APPLICABILITY

In the field of ophthalmology there is significant interest in multi focal solid lens such as the compound micro lens of the present invention, particularly for lens replacement in cataract surgery. Such lenses would be of considerable utility for cornea replacement and in the field of contact lenses.

1. A compound micro lens, comprising:
a substrate shaped to have a first plurality of 25 or more micro-lenses having a first focal length, and a second plurality of 25 or more micro-lenses having a second focal length.

2. The compound micro lens of claim 1 shaped to replace a human eye lens.

3. The compound micro lens of claim 1 wherein said first focal length is a negative focal length and said second focal length is a positive focal length.

4. The compound micro lens of claim 1 further including side fixture elements attached to said substrate, said side fixture elements being shaped to hold said compound micro lens in place.

5. The compound micro lens of claim 1 in which said first focal length is selected to enables a patent to read.

6. The compound micro lens of claim 5 wherein said second focal length is selected to enable a patent to drive a car.

7. The compound micro lens of claim 1 further comprising a cover material.

8. The compound micro lens of claim 7 wherein said cover material as a different refractive index from said substrate.

9. The compound micro lens of claim 1 wherein said first and second plurality of micro-lenses are arranged in concentric rings.

10. The compound micro lens of claim 1 wherein said first and second plurality of micro-lenses comprise hexagons.
